(19) United States
(12) Patent Application Publication
(10) Pub. No.: US 2018/0317794 A1
(43) Pub. Date: Nov. 8, 2018

(54) SYSTEM AND METHOD FOR DETECTING AND MEASURING BIOSIGNALS

(71) Applicant: Emotiv, Inc., San Francisco, CA (US)

(72) Inventors: Geoffrey Ross Mackellar, Elanora Heights (AU); Tan Le, San Francisco, CA (US)

(21) Appl. No.: 15/970,583

(22) Filed: May 3, 2018

Related U.S. Application Data
(60) Provisional application No. 62/576,390, filed on Oct. 24, 2017.
(60) Provisional application No. 62/500,856, filed on May 3, 2017.

(51) Int. Cl. A61B 5/0478; A61B 5/0488; A61B 5/048; A61B 5/00

(52) U.S. Cl. CPC A61B 5/0478; A61B 5/0488; A61B 5/0245; A61B 5/6814; A61B 5/7203; A61B 5/048

(57) ABSTRACT

An embodiment of a system for detecting and measuring biosignals preferably includes a biosignal sensor subsystem comprising a set of sensors configured to detect biosignals from the user, a sensor interface for pre-processing signals from the set of sensors and an electronics subsystem coupled to the biosignal sensor subsystem, the electronics subsystem configured to power the system and facilitate processing of biosignals detected by the system. An embodiment of a method for detecting and measuring biosignals of a user preferably includes inserting a payload into tissue of a user, navigating the payload through tissue of the user, and securing the payload to the user.

### CROSS-REFERENCE TO RELATED APPLICATIONS

This application claims the benefit of U.S. Provisional Application No. 62/576,390 filed 24 Oct. 2017 and U.S. Provisional Application No. 62/500,856 filed 3 May 2017, each of which is incorporated in its entirety by this reference.

### TECHNICAL FIELD

This invention relates generally to the biosignals field, and more specifically to a new and useful system and method for detecting and measuring biosignals in the biosignals field.

### BRIEF DESCRIPTION OF THE FIGURES

FIG. 1 depicts a schematic of an embodiment of a system for detecting and measuring biosignals.

FIG. 2 depicts a variation of a system for detecting and measuring biosignals.

FIG. 3 depicts a variation of a system for detecting and measuring biosignals.

FIG. 4 depicts a variation of a sensor having a retention mechanism.

FIG. 5 depicts a variation of a portion of a system for detecting ahd measuring biosignals.

FIG. 6 depicts a variation of a method for inserting a system for detecting and measuring biosignals.

FIG. 7 depicts a variation of a portion of a method for inserting a system for detecting and measuring biosignals.

### DESCRIPTION OF THE PREFERRED EMBODIMENTS

The following description of the preferred embodiments of the invention is not intended to limit the invention to these preferred embodiments, but rather to enable any person skilled in the art to make and use this invention.

### 1. Overview

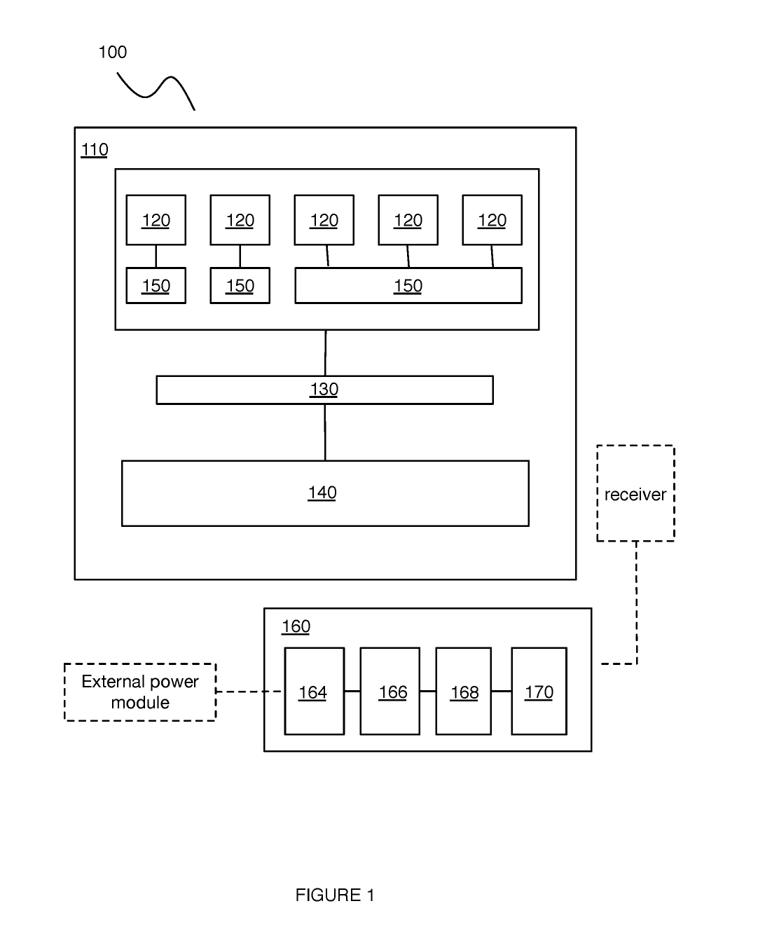

As shown in FIG. 1, an embodiment of a system 100 for detecting and measuring biosignals of a user comprises: a biosignal sensor subsystem 110 comprising a set of sensors 120 configured to detect biosignals from the user; a sensor interface 150 configured to pre-process signals from the set of sensors 120; and an electronics subsystem 160 coupled to the biosignal sensor subsystem 110 and configured to power the system 100 and facilitate processing of biosignals detected by the system 100. The system 100 can further include any or all of: a retention mechanism 140; a system insertion element (e.g., surgical tool, needle, sheath, etc.); a supplementary sensor system; and any other suitable element.

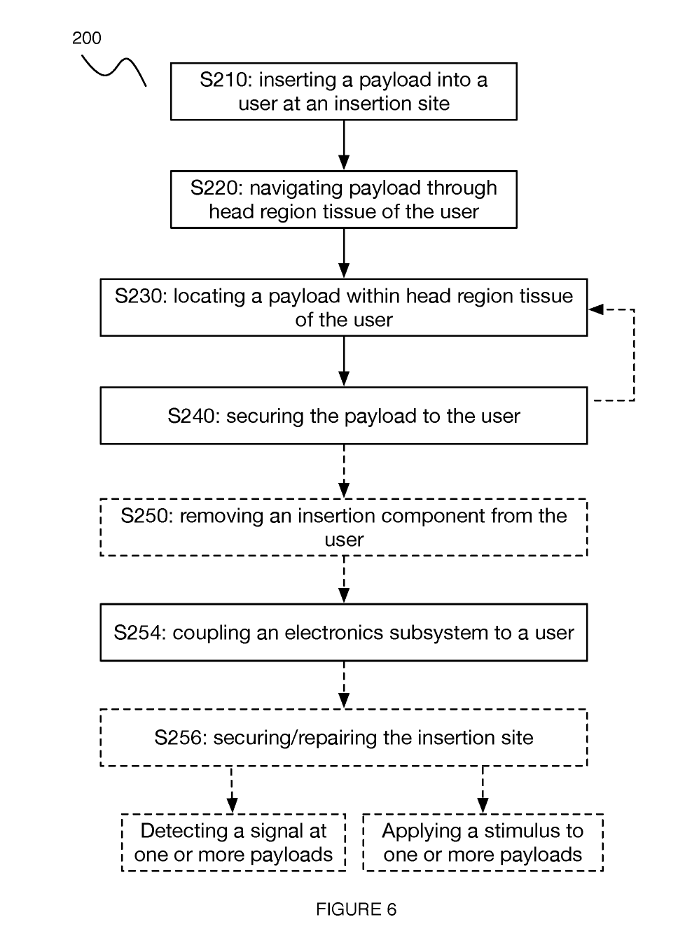

As shown in FIG. 6, an embodiment of a method 200 for detecting and measuring biosignals of a user comprises: inserting a payload into tissue of a user S210; navigating the payload through tissue of the user 220; and securing the payload to the user S240. The method 200 can further include any or all of: locating the payload within tissue of the user S230; removing a temporary component from the user S250; placing an electronics subsystem S254; securing and/or repairing an insertion site of the user S256; detecting one or more biosignals S260; processing one or more biosignals 270; and any other suitable step. The method 200 can be used in conjunction with system 100 and/or any other suitable system.

The systems and methods described below can include and/or be used with any or all of the systems and methods of U.S. application Ser. No. 13/565,740 filed 2 Aug. 2012, U.S. application Ser. No. 13/903,806 filed 28 May 2013, U.S. application Ser. No. 15/835,952 filed 8 Dec. 2017, U.S. application Ser. No. 13/903,832 filed 28 May 2013, U.S. application Ser. No. 15/683,581 filed 22 Aug. 2017, U.S. application Ser. No. 13/903,861 filed 28 May 2013, U.S. application Ser. No. 14/447,298 filed 30 Jul. 2014, U.S. application Ser. No. 14/447,326 filed 30 Jul. 2014, U.S. application Ser. No. 15/058,622 filed 2 Mar. 2016, and U.S. application Ser. No. 15/209,582 filed 13 Jul. 2016, each of which is incorporated in its entirety by this reference. However, the system and/or methods can be used with any other suitable set of systems and methods.

### 2. Benefits

Variations of the system and method can afford several benefits and/or advantages.

First, variations of the system and method in which the set of sensors are subcutaneously coupled to the user achieve improved electrical contact quality and noise reduction compared to sensors coupled to the external surface of the user. In specific examples, each sensor can achieve a greater than tenfold decrease in EEG signal noise versus an externally coupled EEG sensor.

Second, variations of the system and method in which the sensors are encapsulated by a housing configured to couple to the surrounding tissue can significantly reduce undesired movements (e.g., slippage, migration, etc.) of sensors relative to external surfaces of the user, which can otherwise degrade performance of the sensors. Insertion (e.g., subcutaneously, transcutaneously, etc.) of the sensors of such variations can be performed via threading of elongated sensor-carrying members (e.g., threads, sutures, thread-shaped wires, etc.) between the skin and subcutaneous tissue of a user's scalp, to position sensors attached thereupon at the desired location proximal to the brain of the user. Insertion is preferably minimally invasive, low-risk, and readily performed without hospitalization (e.g., be an outpatient procedure, be an at-home procedure); however, insertion can be otherwise suitably performed.

Third, variations of the system and method can enable headwear (e.g., hats, headbands, headphones, etc.) to be comfortably worn by the user without mechanical interference by the system, since subcutaneous components do not protrude from the user's body (e.g., from the user's head). For example, a user can easily wear a baseball cap or beanie while using such variations of the system. In another example, the user can assume one of various common sleeping positions (e.g., on his/her side, on his/her back, on his/her stomach, etc.) without mechanical interference between the system and a pillow or other sleeping implement of the user. As such, variations of the system and method can support monitoring of cognitive and/or physiological states of the user in scenarios not readily available to other wearable devices. In another example, a variation of the system and method can couple to a disabled user without mechanically interfering with a head support of the disabled user's assistance device (e.g., a powered wheelchair including a head support).

Fourth, variations of the system and method can enable discrete coupling of sensors to a user's body (e.g., subcutaneously), and discrete placement of external (e.g., non-subcutaneous) system components (e.g., at the base of a head region of the user) to minimize the externally visible system components, which could otherwise be unstylish and/or unsightly.

Fifth, variations of the system and method can enable a user to communicate with a computing system directly using biosignals (e.g., EEG signals). For example, the system can provide a brain machine interface (BMI) and/or brain computer interface (BCI) between a user and a computing system (e.g., an active electromechanical prosthetic, an augmented-reality or virtual-reality system, etc.). In some variations of the system, the system can be coupled to a disabled user, wherein the system functions to control a secondary system (e.g., a prosthetic/wearable), interface with the internet (e.g., search engine), or assist with any other task or goal.

Sixth, variations of the system and method can improve signal to noise ratio of biosignal acquisition, because signals can be acquired in a low-impedance region below the skin (e.g., compared to above the skin), avoiding contact conditions through hair or in challenging environments. For example, sensors can be operated in a continuously-operating mode, wherein signals are constantly and/or near-constantly acquired at the sensor.

Seventh, variations of the system and method can be readily adapted to include various sensing modes including emotional, facial and BCI detections and biometric identification, as well as other sensing modes such as motion, electrocardiography, electromyography, blood chemistry and any other suitable sensing modes.

Eighth, variations of the system and method can implement electrostimulation (e.g., neurostimulation) such as transcranial direct current stimulation (tDCS), transcranial alternating current stimulation (tACS), and any other suitable form of electrostimulation using the same subcutaneously-positioned electrode architecture. In related variants, the same sensors can be used for neurostimulation by delivering current in lieu of or in addition to measuring biosignals (e.g., collecting current).

Ninth, variations of the system and method can be configured for sensor insertion at any body location, for any suitable purpose. For example, variations of the system and method can be configured for subcutaneous insertion into an extremity of a user (e.g., hand, foot, finger, toe, etc.) and can include a blood glucose sensor for continuously monitoring blood glucose at a body region wherein blood glucose readings are maximally temporally salient (e.g., recent). In another example, the system can be integrated into sutures and used for monitoring wound healing during and after surgical operations (e.g., monitoring blood oxygenation, tissue health, etc.). In another example, an embodiment of the system and method can be used to embed a long-term multi-lead heart sensing system (e.g., ECG or EKG) within a patient. In yet another example, an embodiment can be used to implant sensors close to muscle groups in specific regions (e.g., the forearm of a user, the calf of a user, etc.), to enable direct sensing of motor activity (e.g., motor neuron activity, muscle activation, etc.) without a transdermal physical connection. Related example variations can be used for any suitable subcutaneous sensing of any suitable biological parameter.

Tenth, variations of the system and method can function to provide an optimal (e.g., user-specific, adjustable, etc.) fit to a user, wherein the optimal fit functions to detect a set of desired signals, accurate signals, consistent signals over time, or any other signals.

Eleventh, variations of the system (e.g., subcutaneous, transcutaneous, topical, etc.) and method can function to reduce and/or eliminate risks and side effects of invasive neural procedures (e.g., deep brain stimulation electrode implantation).

However, the system and method, and variations thereof, can otherwise afford any suitable benefits and/or advantages.

### 3. System

The system 100 functions to provide a biosignal sensing tool for a user, a group of users, or an entity associated with the user/group of users, in a format that is coupled to a user's body (e.g., subcutaneously). Thus, the system 100 is preferably configured to couple to the user(s) as the user(s) perform activities (e.g., watching videos, receiving stimuli, exercising, reading, playing sports) in his/her daily life. The variations and examples of the system 100 can be implemented alone, in combination, or in any other arrangement.

Preferably, the biosignals detected and measured by the system 100 comprise bioelectrical signals; however, the biosignals can additionally or alternatively comprise any other suitable biosignal data. In variations of the system 100 for bioelectrical signal detection and measurement, the system 100 is preferably configured to detect electroencephalograph (EEG) signals, which can be reflective of cognitive, mental, and affective state of the user. However, the bioelectrical signals can additionally or alternatively include any one of more of: signals related to magnetoencephalography (MEG) impedance or galvanic skin response (GSR), any magnetic or electromagnetic signals, electrocardiography (ECG), heart rate variability (HRV), electrooculography (EOG), and electromyelography (EMG). Other variations of the system 100 can additionally or alternatively comprise sensors (e.g., supplementary sensors) configured to detect and measure other biosignals, including biosignals related to cerebral blood flow (CBF), optical signals (e.g., eye movement, body movement), mechanical signals (e.g., mechanomyographs) chemical signals (e.g., blood oxygenation), acoustic signals, temperature, respiratory rate, positional information (e.g., from a global positioning sensor), motion information (e.g., motion information-such as the detection of a user fall—from an accelerometer and/or a gyroscope with any suitable number of axes of motion detection), and/or any other signals obtained from or related to biological tissue or biological processes of the user, as well as the environment of the user. Positional information can, for example, provide information to an emergency response team in the event that an adverse mental condition (e.g., a seizure) is detected at the system 100. Furthermore, motion information can enable determination of user gait, activity, tremors, and other details pertinent to the diagnosis or characterization of the user's situation, and can additionally or alternatively facilitate correction of and/or compensation for motion artifacts in biosignals detected at the system 100.

The system 100 is preferably configured to be coupled subcutaneously to a user, require little maintenance of subcutaneously-coupled system components, and maintain contact between the set of sensors and the user as the user performs activities in his/her daily life. As such, the system 100 is preferably comfortable for long term use, adapted for long-term subcutaneous positioning of sensors without requiring unduly invasive procedures for insertion or removal of subcutaneous components, includes sufficient power storage, and adapts in response to the user's motions, in order to avoid undue subcutaneous migration (e.g., greater than 10-15 mm) of the set of sensors with respect to the user. The subcutaneously-inserted portions of the system 100 (e.g., the set of sensors) can be inserted at any suitable subcutaneous position (e.g., liminal between the skin and subcutaneous tissue, within the subcutaneous tissue, liminal between the subcutaneous tissue and muscle tissue, etc.). The system 100 can be placed in regions of low stiffness (e.g., placed in region having an elastic modulus of less than 200 kPa, less than 100 kPa, less than 50 kPa, between 22 and 102 kPa, less than 10 kPa, less than 5 kPa, greater than 3 kPa, etc.), placed in a region of high stiffness (e.g., elastic modulus greater than 200 kPa), or any other suitable region. Additionally or alternatively, the system 100 can be configured in any suitable manner (e.g., coupled transcutaneously, coupled to an external surface of the user, etc.) that enables detection and/or measurement of biosignals of the user.

### 3.1 System—Biosignal Sensor Subsystem

The biosignal sensor subsystem 110 functions to detect one or more biosignals (e.g., EEG signals, gamma waves, alpha waves, beta waves, theta waves, delta waves, etc.) from a user. Additionally or alternatively, the biosignal sensor subsystem 110 can function to provide stimulation to a user, collect data for a secondary use (e.g., to control a secondary system, compare to data from a second user, compare to prior dataset of user, etc.), or perform any other suitable function.

The biosignal sensor subsystem 110 is preferably at least partially arranged subcutaneously in a head region of the user (e.g., between skin and skull of user's head, between skin and connective tissue, within connective tissue, within epicranial aponeurosis, within loose areolar tissue, within periosteum/pericranium, etc.), but can additionally or alternatively be arranged transcutaneously (e.g., within skin of scalp, within skin and connective tissue, within skin and connective tissue and aponeurosis, etc.), external to the scalp (e.g., adhered to an outer layer of the scalp skin, embedded in an outer layer of the scalp skin, etc.), within or on a neck region of the user, within or on a face region of the user, within or on any other body part of the user, within the skull (e.g., placed on the cortical surface, embedded in brain tissue, etc.) remote from the user, and/or arranged in any other way with respect to the user.

The head region preferably correlates to (e.g., is arranged proximal to, over, adjacent to, partially over, within, etc.) a predetermined brain and/or skull region of a user, such as an anatomical brain structure (e.g., cortex, cerebellum, etc.), a brain lobe (e.g., frontal lobe, parietal lobe, temporal lobe, occipital lobe), a skull region (e.g., frontal bone, occipital bone, etc.), a surface feature of the brain (e.g., specific fold, central sulcus, etc.) but can additionally or alternatively refer to a region of the neck, face, and/or any other part of the user's body.

In a first variation, the biosignal sensor subsystem 110 is partially subcutaneous and partially transcutaneous (e.g., sensors 120 arranged subcutaneously but coupling system 130 arranged transcutaneously and/or external to the user, coupling system 130 arranged subcutaneously and sensors 120 arranged transcutaneously, etc.). In other variations, the biosignal sensor subsystem 110 can be arranged partially subcutaneously and partially externally (e.g., on the skin of a user), partially transcutaneously and partially externally, and/or in any number and combination of arrangements and locations.

In a specific example, the biosignal sensor subsystem includes one or more barbed threads or barbed sutures (e.g., similar to those used for barbed suture lifts or plastic surgery) that each include one or more on-thread or on-suture sensors (e.g., electrodes, etc.); an electrical terminal (e.g., at one or both ends of the thread); and/or electrical connectors (e.g., wires) extending between the sensor(s) and the electrical terminal, the sutures and the electrical terminal, or between any other suitable component. However, the barbed threads or barbed sutures can include any other suitable component.

The system 100 preferably includes a single biosignal sensor subsystem 110 but can additionally or alternatively include multiple biosignal sensor subsystems 110 (e.g., within a single head region, over multiple head regions, separate biosignal sensor subsystems for separate head regions, etc.). In variations having multiple biosignal sensor subsystems 110, a subset of the subsystems can be connected (e.g., electrically connected, converge at a multiplexer, communicatively coupled to a single user device), all connected, or all separate (e.g., distinct processing system for each biosignal sensor subsystem, a distinct electronic subsystem for each biosignal sensor subsystem), or otherwise related.

The biosignal sensor subsystem 110 can operate in any number of operation modes, such as but not limited to: on/off operation modes; various detection operation modes (e.g., corresponding to a parameter such as: a particular sensor; a particular brain region; a particular signal parameter such as an amplitude or frequency; etc.) wherein the biosignal sensor subsystem 110 is actively detecting a biosignal; various stimulation operation modes (e.g., corresponding to parameters such as the detection operation mode parameters) wherein the biosignal sensor subsystem 110 is actively applying a stimulus; and any other suitable operation mode(s). The set of operation modes can be predetermined (e.g., detect at the same time every day, turn on at 8 AM and off at 8 PM, etc.) or pre-programmed, and/or determined based on any or all of: input from onboard sensors, input from supplementary sensors (e.g., location, temperature, moisture, another body signal such as heart rate, onset of an event, etc.), learned behavior, or any other suitable trigger.

### 3.2 System—Set of Sensors 120

The biosignal sensor subsystem 110 preferably includes a set of one or more sensors 120, which individually or collectively function to detect and/or measure one or more biosignals from a user. Additionally or alternatively, one or more of the set of sensors 120 can function to provide one or more input signals to a user, serve as a reference (e.g., common mode sensor (CMS), sensor associated with a driven right leg (DRL) module, etc.) to another sensor (e.g., electroencephalography (EEG) sensor), or perform any other suitable function. The set of sensors 120 can be active (e.g., require a power source, be physically or wirelessly coupled to a power source, etc.), passive (e.g., not require a power source, have no coupling to a physical power source, etc.), or any combination of active and passive. The sensors 120 are preferably electrodes, but can additionally or alternatively include: optical sensors, such as light sensors and cameras; orientation sensors, such as accelerometers, gyroscopes, and altimeters; audio sensors, such as microphones; temperature sensors; or any other suitable sensor. The sensors can be internally arranged within the user (e.g., transcutaneous, subcutaneous, etc.), partially arranged within the user (e.g., embedded in the skin of a user), arranged external to the user (e.g., on a skin surface, uncoupled from a user, remotely located, etc.), or arranged in any combination of arrangements.

The set of sensors 120 is preferably arranged on or in a coupling subsystem 130 but can additionally or alternatively be arranged separate from or in absence of a coupling subsystem (e.g., in a wireless sensor network). The set of sensors can be defined by a coupling subsystem 130 (e.g., defined by an exposed contact region; defined by a coating, such as an electrically insulative coating; defined by an absence of coating, etc.), adhered to a coupling subsystem 130 (e.g., with an adhesive, solder, etc.), wirelessly coupled to a coupling subsystem, fastened to a coupling subsystem 130, spread among multiple coupling subsystems 130, or arranged in any other suitable way.

In a first variation, the set of sensors 120 are mounted to discrete locations on the coupling subsystem (e.g., evenly spaced locations, unevenly spaced (e.g., user-specific, anatomy-specific, etc.) locations, in a one-dimensional array, in a two-dimensional array, etc.). In a specific example, the set of sensors 120 is distributed among a series of branching coupling subsystems 130 extending radially from an electronics subsystem 160. In a second specific example, the sensors 120 are connected to a coupling subsystem 130 in a mesh arrangement, such as a mesh that spans multiple head regions. In a second variation, the set of sensors 120 are removably mountable (e.g., removable coupling) and/or adjustable (e.g., sliding, linkable, configured to grow with user, configured to reach specific anatomical components, etc.) with respect to a coupling subsystem 130. In a first specific example, the coupling subsystem 130 can be connected to the set of sensors 120 after installation (e.g., implantation in a user). In a second specific example, the coupling subsystem 130 can be connected to the set of sensors 120 before installation. In a third example, the set of sensors 120 are parts of a wireless sensor network and have no connection to a coupling subsystem 130.

During installation of the set of sensors 120, the set of sensors 120 can be arranged at one or more head regions within or on a user, proximal to a head region of the user (e.g., at an offset from a head region) or within or on any other body part of the user. The sensors 120 can be configured to be installed at a subcutaneous depth in the scalp of the user (e.g., depth greater than 1,450 microns, greater than 5,000 microns, greater than 10,000 microns, between 5,000 and 10,000 microns, etc.), but can additionally or alternatively be arranged at a transcutaneous depth (e.g., between 1,450 and 5,350 microns), on/in an external surface (e.g, on external surface of scalp skin, neck skin, etc.), or at any other suitable location. The sensors 120 are all preferably arranged at substantially the same depth (e.g., all subcutaneous) but can alternatively be arranged at different depths with respect to each other.

Each of the set of sensors 120 is preferably individually indexed; additionally or alternatively, subsets of sensors 120 can be individually indexed, all of the set of sensors 120 can be indexed together, or any other arrangement of sensors can be indexed in any suitable way. The set of sensors 120 can be connected in series, in parallel, wirelessly connected, not connected at all, or arranged in any suitable way. The sensors can be connected to the same data bus, to different data busses (e.g., individual data buses for each sensor, shared data busses for different sensor sets, etc.), or otherwise connected. In a first variation, the set of sensors 120 are connected in series, share a common bus and/or wire (e.g., wire of a coupling subsystem 130), and are individually indexed. In a second variation, the set of sensors 120 are connected in parallel to a sensor interface (e.g., electronics subsystem 160). In a third variation, all of the sensors in a predetermined head region (e.g., motor cortex) are indexed together.

Each of the sensors 120 can have any suitable shape (e.g., square, round, etc.), thickness (e.g., smaller than the thickness of the surrounding tissue region, equal to the thickness of the surrounding tissue region, etc.), width, and length (e.g., between 2 and 3 millimeters (mm), less than 2 mm, greater than 3 mm).

The set of sensors 120 preferably includes electrically conductive regions that provide low to moderate contact impedances (e.g., less than 10,000 ohms, less than 5,000 ohms, less than 1,000 ohms, less than 400 ohms, less than 100 ohms, greater than 1 ohm, etc.) and low-voltage signal transmission. The sensors of the set of sensors 120 are also preferably low-noise (e.g., signal-to-noise ratio greater than 40 decibels [dB], greater than 25 dB, greater than 10 dB, greater than 5 dB, between 25 and 40 dB, etc.), and/or provide non-polarizable contact with the user's tissue (e.g., subcutaneous tissue). As such, the sensors preferably behave such that the contact half-cell voltage is independent of current magnitude or direction of flow in a particular range of interest. However, the sensors 120 can alternatively comprise sensors with any suitable noise-handling and/or polarizability behavior. The conductive regions are preferably constructed from a conductive material (e.g., stainless steel, nickel, iron, cobalt, tungsten, titanium, copper, gold, etc.) resistant to corrosion and suitable for semi-permanent emplacement within human tissue. However, the conductive regions of the set of sensors 120 can be characterized by any other suitable characteristic(s).

Each sensor preferably defines an exposed contact region that is configured to detect biosignals (e.g., EEG signals) from the vicinity of the sensor. The exposed region is preferably made of biocompatible material(s) such as titanium, stainless steel, and/or any other suitable conductor. In some variations, the exposed region and/or any other region of the sensor 120 can be coated in an implantable polymer (e.g., poly(3,4-ethylenedioxythiophene)/PEDOT), which can improve electrochemical stability (e.g., to reduce signal noise). However, the exposed contact region can additionally or alternatively be uncoated, or otherwise suitably coated. The exposed contact region is preferably between 2 and 3 mm in length, but can additionally or alternatively have any suitable length and/or size (e.g., greater than 1 mm in length, less than 3 mm, greater than 3 mm, etc.).

The set of sensors 120 preferably includes one or more reference sensors (e.g., CMS, sensors associated with a DRL module, etc.), such as those described in U.S. application Ser. No. 15/209,582 filed 13 Jul. 2016, which is incorporated in its entirety by this reference. Additionally or alternatively, the system 100 can include any other suitable reference sensor(s), omit specific reference sensors and self-reference the bioelectrical potential measurement (e.g., against an arbitrary reference value, a predetermined reference value, an electrical potential value detected at another EEG sensor, etc.), or otherwise suitably obtain a differential potential measurement.

In one variation, the system 100 includes one or more supplementary optical sensors (e.g., broadband optical detector, narrow band optical detector, photodiode, etc.) and/or optical sources (e.g., light source, broadband optical source, narrow band optical source, laser, light emitting diode, superluminescent light emitting diode, etc.), which can be configured to detect or measure a characteristic (e.g., tissue characteristic, endogenous substance, flow rate, etc.) of the user, such as through differential absorption methods. The system 100 can, for instance, include a pulse oximeter system configured to measure an oxygen level of the user. Additionally or alternatively, the system 100 can apply one or more backscatter techniques (e.g., near infrared scattering) configured, for instance, to measure a change in oxygenation within or between one or more brain regions of the user. Further additionally or alternatively, the system 100 can include any other optical system or combination of optical systems configured for any suitable purpose.

In one specific example, for instance, the system 100 includes one or more internal light sources (e.g., arranged subcutaneously, transcutaneously, proximal to brain tissue, etc.), wherein detection of light from the light sources by one or more sensors (e.g., implanted sensors, external sensors, etc.) determines a measurement (e.g., tissue type, tissue thickness, etc.) of intervening tissue between the light source(s) and light sensor(s).

In another variation, the set of sensors 120 comprises a first anterior frontal sensor subset, a second anterior frontal sensor subset, a first temporal lobe sensor subset, a second temporal lobe sensor subset, a central sensor subset, and a common mode sensor subset to provide a reference signal. In the example, each sensor in the set of sensors provides a single channel for signal detection, is characterized by a frequency bandwidth from above DC to a threshold frequency (e.g., 80 Hz, 100 Hz, 120 Hz, above 120 Hz, etc.), and is characterized by a nominal voltage of 0-100 microvolts; however, the full dynamic range of the electronics system in the example can accommodate 5 millivolt signals to accommodate large electromyographic signals (e.g., eye blinks, clenched jaw signals). Furthermore, in the example, the set of sensors is configured to be non-adjustable in location (e.g., subcutaneously embedded at a particular position between the user's skin and skull), while providing adequate signal detection from multiple regions of the brain. In other variations, the set of sensors 120 can comprise any suitable number of sensors in any suitable configuration for detecting biosignals from the user.

The set of sensors 120 can include any suitable number of sensors, such as a single sensor (e.g., configured to measure signals from a particular head region, such as a head region associated with a user pathology) or multiple sensors (e.g., sensors spread over a coupling subsystem 130, sensors configured to detect signals from multiple head regions, etc.). In a first variation, the set of sensors includes 256 individual sensors (e.g., 256 single-channel sensors) forming a high density array. In related examples, however, the set of sensors can include any suitable number of sensors (e.g., 1, 10, 32, 64, etc.).

The set of sensors 120 functions to directly detect biosignals (e.g., bioelectrical signals) from a user, wherein each sensor in the set of sensors 120 is preferably configured to provide at least one channel for signal detection. Additionally or alternatively, a single channel can be shared among multiple sensors 120 (e.g., multiple sensors corresponding to a particular head region, all the sensors 120, etc.). Preferably, each sensor in the set of sensors 120 is identical to all other sensors in composition; however, each sensor in the set of sensors 120 can be non identical to all other sensors in composition, in order to facilitate unique signal detection requirements at different region of the user's body (e.g., user's brain). The set of sensors 120 can comprise sensors that are non-identical in morphology, in order to facilitate application at different body regions; however, the set of sensors 120 can alternatively comprise sensors that are identical in morphology. The set of sensors 120 can be placed at specific locations on the user, in order to detect biosignals from multiple regions of the user. Furthermore, the sensor locations can be adjustable, such that the set of sensors 120 is tailorable to each user's unique anatomy. Alternatively, the sensor system can comprise a single bioelectrical signal sensor configured to capture signals from a single location, and/or can comprise sensors that are not adjustable in location.

In a first variation, the sensor subsystem 120 includes a series of sensors defined by exposed contact regions of a coupling subsystem 130. In a first specific example, the exposed contact regions are arranged in a series of one-dimensional array along branches of a coupling subsystem 130, wherein the branches all converge at an electronics subsystem 160. In a second specific example, the biosignal subsystem 160 includes 256 exposed contact regions forming a sensor mesh.

In a second variation, the biosignal sensor subsystem 110 includes a set of wireless sensors.

In a third variation, the biosignal sensor subsystem 110 includes all passive sensors.

In a fourth variation, the biosignal sensor subsystem 110 includes all active sensors.

In a fifth variation, the system 100 includes a series of exposed contact regions, each of the series of exposed contact regions configured as an EEG sensor, as well as one or more supplementary sensors (e.g., optical sensor, accelerometer, etc.). The supplementary sensors can be formed from exposed contact regions, be adhered to a coupling subsystem (e.g., soldered to a coupling subsystem), comprise one or more traces on a PCB (e.g., flex printed circuit), or be arranged in any other way.

### 3.3 System—Coupling Subsystem 130

The biosignal sensor subsystem 110 preferably includes one or more coupling subsystems 130, which individually or collectively function to electrically connect one or sensors 120 to an electronics subsystem 160. Additionally or alternatively, the coupling subsystem 130 can function to mechanically connect one or more sensors 120 to the electronics subsystem 160, electrically and/or mechanically connect two or more sensors to each other, retain and/or define a retention mechanism 140 proximal to a sensor 120, to retain the sensors relative to a user's skin, and/or perform any other suitable function.

The system can include one or more coupling subsystems 130. Each coupling subsystem 130 can be associated with one or more sensors or other payload components. The coupling subsystem 130 can be connected to one or more sensors 120, define one or more sensors, or be connected to or define any other element of the system 100. The coupling subsystem 130 can connect two or more sensors together, one or more sensors to an electronics subsystem, or establish an electrical connection between any other elements of the system 100. The coupling subsystem is preferably connected to an onboard electronics subsystem but can additionally or alternatively be connected to an offboard electronics subsystem, any other part of the system 100, any part of the user, and/or any other element inside or external to the system 100.

The coupling subsystem 130 can have a predetermined diameter (e.g., 10 mm; less than 20 mm; less than 10 mm; less than 5 mm; less than 2 mm; less than 1 mm; between 1 mm and 5 mm; less than a diameter of an insertion site; less than a diameter of an insertion tool such as cannula, sheath, needle, spinal tap, etc.; greater than a diameter of an insertion tool such as cannula, sheath, needle, spinal tap, etc.; a diameter determined to minimize injury and/or invasiveness of the insertion procedure; etc.), a fixed diameter, an adjustable diameter, a variable diameter, or any other suitable diameter.

In some variations, the coupling subsystem 130 is constructed to have similar mechanical properties (e.g., stiffness, elastic modulus, flexibility, spring constant, length, diameter, etc.), material properties (e.g., material composition, coating, etc.), and/or electrical properties (e.g., conductivity) as one or more surgical tools or implantable materials (e.g., suture, needle, cannula, sheath, catheter, etc.), which can have any or all of the benefits of: physician familiarity for increased ease-of-use, physician familiarity for device insertion, minimized potential bodily rejection, and/or any other benefit. Examples of the coupling subsystem 130 can include: a set of threads or wires, a mesh, a scaffold, a matrix (e.g., polymer matrix, gel matrix), or be any other suitable temporary or permanent support system.

The coupling subsystem 130 can include any number of a series of navigation features, which can be configured to improve navigation (e.g., straight insertion, sinous insertion, etc.) of the coupling subsystem through tissue of the user, such as, but not limited to: a curvature or series of curvatures along a length (e.g., curved hook end, series of curvatures for sinuous insertion), one or more relatively stiff (e.g., elastic modulus greater than 0.1 giga-pascals [GPa], greater than 3 GPa, greater than 10 GPa, greater than 50 GPa, greater than 100 GPa, greater than 1000 GPa, etc.) regions and/or one or more relatively flexible (e.g., elastic modulus less than 100 GPa, less than 10 GPa, less than 1 GPa, etc.) regions (e.g., relatively stiff end adjacent to a relatively flexible region, series of alternating stiffnesses along length, etc.), a variable diameter (e.g., pointed end, one or more protrusions along length, one or more indents along length, etc.), and/or any other features configured for navigation and/or retention (e.g., during or post-navigation) of the coupling subsystem.

The coupling subsystem 130 is preferably at least partially constructed from a conductive material (e.g., copper, steel, gold, silver, aluminum, brass, titanium, conductive polymer, carbon rubber, etc.) to establish one or more electrical connections (e.g., with a sensor 120, with an electronics subsystem 160, etc.). The coupling subsystem 130 is further preferably at least partially constructed from a biocompatible material (e.g., fully biocompatible material, a biocompatible coating, etc.). In one variation, the coupling subsystem 130 includes one or more conductive wires (e.g., power wire, sensor wire, etc.). In a second variation, the coupling subsystem 130 can additionally or alternatively include one or traces of a printed circuit board (PCB).

The coupling subsystem 130 can have a single electrically connected coupling subsystem with multiple extensions (e.g., multiple traces, multiple extending wire bundles, multiple extending wires, etc.). Additionally or alternatively, the system 100 can include multiple coupling subsystems 130 (e.g., different coupling subsystems for different head regions, separate coupling subsystem for each sensor or for different groupings of sensors, etc.). In variations having multiple coupling subsystems 130, the coupling subsystems 130 can be electrically isolated (e.g., separated by insultaive connector) or connected (e.g., contiguous, soldered, etc.), communicatively isolated or connected, mechanically connected (e.g., connected with an insulative attachment piece, a conductive attachment piece, soldered, welded, etc.) or isolated, or arranged in any other suitable way.

The coupling subsystem 130 preferably at least partially defines one or more sensors (e.g., exposed contact regions) but can additionally or alternatively be connected to one or more sensors (e.g., welded to, soldered to, threaded/braided with, adhered to, tied to, machined with, etc.). When installed (e.g., implanted in a user), the coupling subsystem 130 can extend in any pattern (e.g., single branch, linear array, two-dimensional array, web, mesh, etc.). The coupling subsystem 130 can be installed at the same depth or a different depth (e.g., at a greater depth) with respect to one or more sensors in the tissue of a head region of the user. In one variation, the set of sensors 120 are arranged at a transcutaneous depth coupling subsystem 130 is arranged on an external surface of the user. In another variation, the set of sensors is arranged at a subcutaneous depth and the coupling subsystem is arranged at a transcutaneous depth. Additionally or alternatively, the coupling subsystem 130 can be arranged in any suitable way with respect to the set of sensors 120 and to a head region of the user.

The coupling subsystem 140 is preferably configured to include slack (e.g., along its length) when installed in a user, which in one variation is measured as a greater length of the coupling subsystem 130 (e.g., wire, wire bundle, etc.) between two points (e.g., between two sensors wherein each of the sensors is connected to the coupling subsystem 130) than a distance between the two points (e.g., shortest distance between the two points; straight distance between the two points; arc length between the two points, such as the arc length defined by a layer of the scalp; etc.). The slack can be removed with retraction of the installation tool, subsequent adjustment (e.g., tightening), left in the installed system, or otherwise managed.

In a first variation, the coupling subsystem 130 includes bundle of wires, which can be any of: twisted, braided, coiled, helically arranged, physically connected, physically separated (e.g., with spacers), electrically connected, electrically separated (e.g., include insulative coating/sleeve), coated (e.g., include a biocompatible coating, insulative coating outside of exposed contact regions, etc.), or arranged in any other way. The wire bundle preferably includes a wire associated with each sensor (e.g., each exposed contact region), as well as power wires (e.g., a positive and negative lead) as shown in FIG. 5. The system also preferably includes a plurality of wire bundles to facilitate distribution of the set of sensors over any desired head region of the user (e.g., the user's scalp, subcutaneous tissue, etc.), which, in a specific example, can extend from a central electronics subsystem 160 in any suitable manner (e.g., in a tortuous fashion, a spiral pattern, a star pattern, a lopsided sunburst pattern, etc.). However, each wire bundle can additionally or alternatively include any suitable wires, and in some variants can omit power wires (e.g., in variants wherein each sensor is powered by a local energy storage device that can be charged in situ). Additionally, the system (e.g., the set of sensors) can include any suitable number of wire bundles.

In an alternative variation, the set of sensors can be printed onto a flexible printed circuit (FPC). For example, the set of sensors can include a set of traces, each trace having a one-to-one correlation with each sensor, printed onto a flexible substrate of any suitable shape. However, the set of traces can have any suitable correlation with each sensor (e.g., each sensor may include two signal traces and two power traces). The FPC is preferably encapsulated by a biocompatible material, in a similar manner to the wire bundle described above, but can additionally or alternatively be un-encapsulated or include any other suitable material. In a specific example, the FPC is a multi-layer printed circuit, wherein the outermost layers are made up of a biocompatible polymer, and the internal layers include conductive portions that make up the conductive traces. The exposed portions of the sensors can, in this example, be formed via etching or other suitable removal of the outermost layers of the FPC. However, the set of sensors can be otherwise suitably constructed by way of and/or including an FPC.

In a third variation, such as a wireless sensor network, the system 100 can have no coupling subsystem 130.

### 3.4 System—Retention Mechanism 140

The biosignal sensor subsystem 110 preferably includes a set of one or more retention mechanisms 140, which functions to mechanically secure one or more sensors 120 to a head region of a user. Additionally the set of retention mechanisms 140 can function to secure one or more coupling subsystems 130 to a head region of a user, one or more sensors 120 to one or more coupling subsystems 130, secure any element of the system 100 to any other element of the system 100, secure any element of the system 100 to any part of the user (e.g., scalp tissue, skin, etc.), reduce migration of the system 100 (e.g., less than 15 mm drift, less than 10 mm drift, less than 5 mm drift, etc.), or perform any other suitable function.

Each of the set of retention mechanisms 140 is preferably connected to the coupling subsystem 130 (e.g., embedded within, adhered to, welded to, manufactured with, wrapped around, etc.). The set of retention mechanisms 140 can be axially aligned with the coupling subsystem 130 (e.g., circumscribed around a bundle of wires), secured to a surface of the coupling subsystem 130, formed from the coupling subsystem 130 (e.g., barbs formed from angled cuts to the coupling subsystem, wire ends extending from the coupling subsystem, barbs extending at an angle from the coupling subsystem [ex. between 0 and 90 degress, less than 20 degrees, greater than 2 degrees], etc.), or coupled in any other suitable way.

Additionally or alternatively, one or more retention mechanisms 140 can be connected to one or more sensors, in the ways described above or in any other suitable way. In some variations, for instance, a retention mechanism 140 can be mounted to any or all of: a sensor interior surface (e.g., proximal to the skull of the user), a sensor perimeter, a sensor exterior surface (e.g., distal to the skull of the user), or at any other part of a sensor 120. One or more retention mechanisms 140 can further additionally or alternatively be connected to any other element of the system 100.

The set of retention mechanisms 140 are preferably configured to engage with (e.g., lock into, puncture, grasp, compress, expand/inflate within, etc.) soft tissue (subcutaneous tissue, fat, skin, brain tissue) of a head region, preferably the head region proximal to (e.g., adjacent to, above, below, etc.) each of the sensors but can additionally or alternatively engage with hard tissue (bone, skull, etc.) of a head region, tissue of a head region different from the head region of the sensor (e.g., head region selected for greater thickness, head region selected for greater thickness, different composition, etc.), within or on a different body part of the user (e.g., transcutaneous lower neck, transcutaneous face, external surface of skin, back, shoulder, etc.), and/or any other part of the user.

Each of the set of retention mechanisms preferably includes one or more protrusions (e.g., spikes, hooks, barbs, etc.) configured to lock into soft tissue. In variations, the barbs can include unidirectional barbs, bi-directional barbs, multi-directional barbs, or any combination. The protrusions can have any length (e.g., barb cut length less than 1 mm, greater than 1 mm, between 1 mm and 3 mm, greater than 5 mm, etc.), diameter (e.g., less than 0.5 mm, greater than 0.5 mm, between 0.25 mm and 2 mm, greater than 2 mm, etc.), variation in diameter (e.g., pointed tip gradually increases to a diameter of 1 mm), barb cut angle (e.g., angle between the tip of the barb and the coupling subsystem less than 45 degrees, less than 20 degrees, greater than 2 degrees, etc.), inter-barb spacing (e.g., between 0.1 mm and 10 mm, greater than 10 mm, etc.), barb base shape (e.g., arcuate barb base), or any other feature or dimension. The set of retention mechanisms 140 can be aligned along a single axis of the coupling subsystem 130, wrapped around or arranged in a helical/spiral configuration around the coupling subsystem 130, circumferentially surrounding the coupling subsystem 130, or arranged in any other way or combination of ways with respect to the coupling subsystem and/or sensors. The set of protrusions are preferably all identical but the set of retention mechanisms 140 can alternatively include multiple different form factors and/or dimensions of protrusions. The protrusion (e.g., barb) direction of each retention mechanism 140 is preferably oriented relative to an insertion direction of the system 100 into the user (e.g., at least partially along with or opposite the insertion direction, so as to not scrape/damage tissue during insertion; normal to the insertion direction (e.g., to achieve stronger engagement) but can additionally or alternatively be oriented relative to one or more terminals of a coupling subsystem 130, one or more sensors 120, or any other element.

The set of retention mechanisms 140 can additionally or alternatively include an expansion feature (e.g., inflatable shell, balloon, expanding and/or flexing barbs [e.g., post deployment from a sheath], etc.) configured to engage with tissue of the user through compression, a biocompatible adhesive, series of stitches, a fastener (e.g., tie), a retention feature configured to engaged with hard tissue (e.g., screw into skull), and/or any other suitable retention feature.

The retention mechanism 140 is preferably configured to be a permanent feature of the system 100, but can alternatively be configured to be biodegradable (e.g., degrades after 1 week, 1 month, 1 year, etc.). The retention mechanism is preferably constructed from a biocompatible material (e.g., polymer, natural polymer, metal, ceramic, etc.) but can additionally or alternatively be constructed from any or all of: a bio-incompatible material (e.g., combined with [e.g., coated in] a biocompatible material), a conductive material (e.g., metal, conductive polymer, carbon rubber, etc.), an insulative material (e.g., polymer, foam, gel, etc.), or any other suitable material. In some variations, the retention mechanism 140 can be configured to incite structural changes in surrounding tissue (e.g., encourage collagen formation and/or stiffening of surrounding structure to increase retention). In one example, for instance, the retention mechanism 140 can be fully or partially biodegradable, wherein structural changes to the surrounding tissue (e.g., collagen formation) during the existence of the retention mechanism persist after the retention mechanism has degraded. In some variations, the retention mechanism 140 can include a drug or medication, wherein the retention mechanism 140 can function to deliver the drug or medication to the user (e.g., to tissue of the user).

The system 100 preferably includes at least one retention mechanism 140 per sensor 120, further preferably multiple retention mechanisms 140 arranged proximal to each sensor (e.g., adjacent to, next to, contiguous with, at an offset from, adhered to, overlaid on, at least partially surrounding a sensor 120, on both sides of sensor 120, etc.). Additionally or alternatively, the system 100 can include a single retention mechanism 140, one or more retention mechanisms 140 biased toward one or more ends of a coupling subsystem 130, one or more retention mechanisms 140 distributed along a length (e.g., along entire length, with a fixed spacing along a length, etc.) of the coupling subsystem 130, or any number of retention mechanisms 140 separate from a coupling subsystem 130, or a set of retention mechanisms 140 arranged in any suitable way.

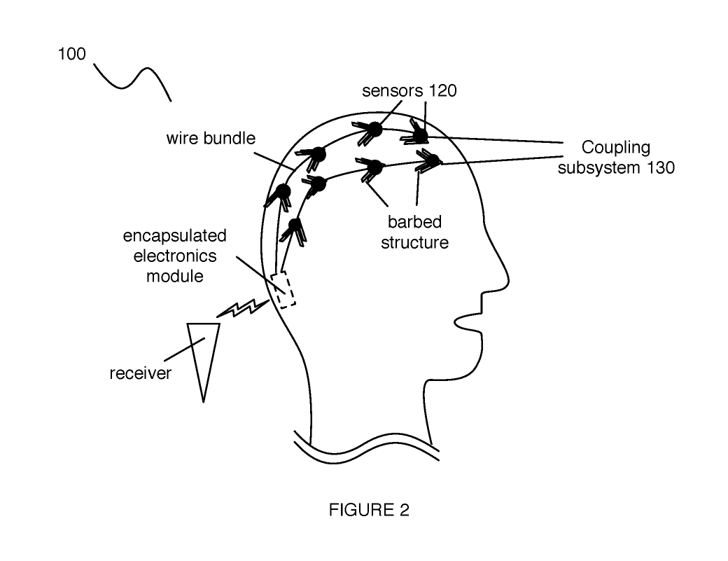

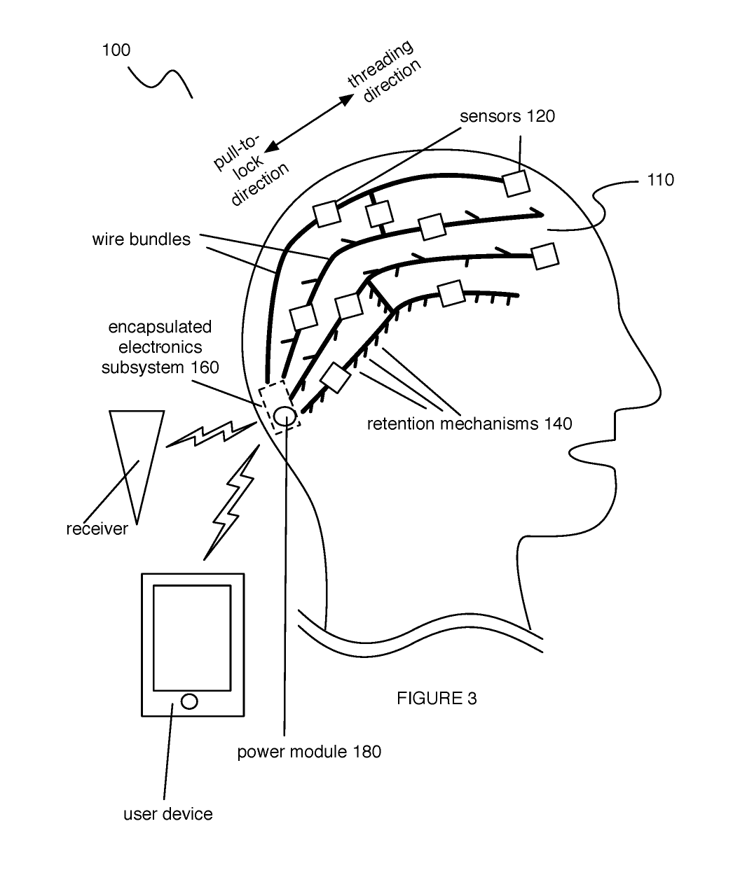

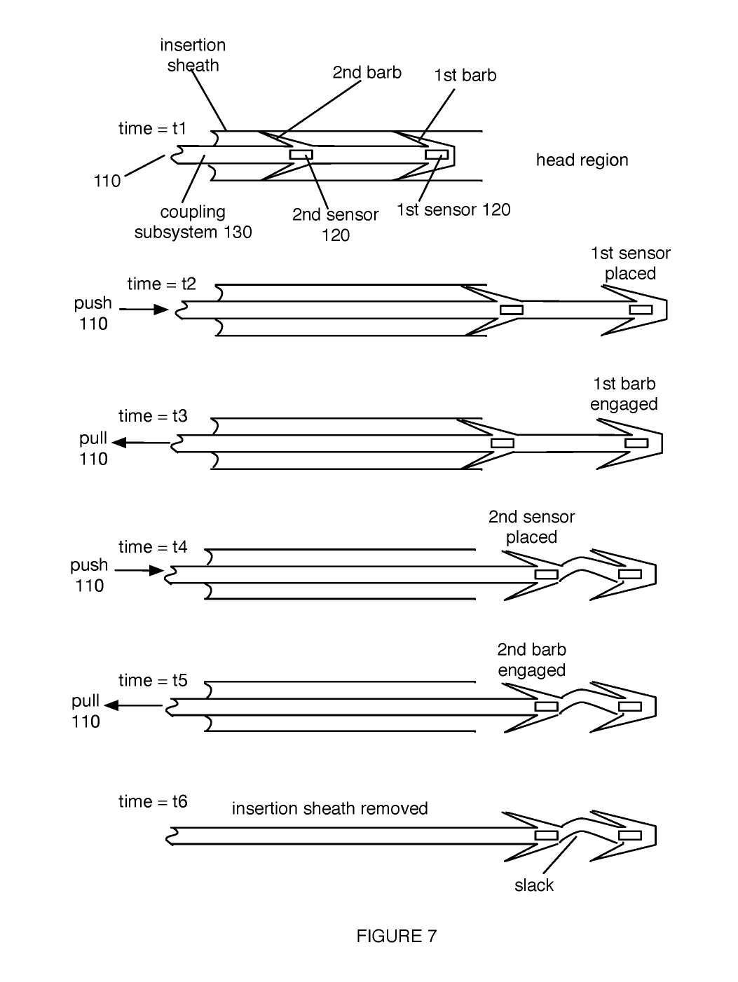

In some variations, (examples shown in FIG. 2 and FIG. 3), the set of retention mechanisms 140 includes a set of barbed structures that promote delivery of the set of sensors within or between tissue layers of the user's body in a first direction, but allow the system to be retained in position once the barbed protrusions are transmitted in a second direction (e.g., a second direction opposing the first direction, as shown in FIG. 7). The barbed protrusions are preferably tapered and have a sharp tip region, but can alternatively have any other suitable morphology. The barbed protrusions can be radially distributed about a defining longitudinal axis (e.g., of wires supporting the set of sensors), or can alternatively be configured relative to sensor supports in any other suitable manner.

In a first specific example, the barbed structures can be configured to retain their morphological aspects over a lifetime of use of the system, such that the barbed structures promote retention of the system in place over a lifetime of use of the system. Alternatively, one or more of the barbed structures can comprise a bioresorbable or otherwise degradable material (e.g., polylactic acid) that resorbs over time. In these variations, fibrous encapsulation of aspects of the system can promote retention of the system in position over time in coordination with degradation/resorption of barbed structures of the system.

In a second specific example, the barbed structures are arranged on an external surface of a sleeve (e.g., flexible sleeve, rigid sleeve, etc.), wherein the sleeve is arranged over at least part of the coupling subsystem.

Additionally or alternatively, variations of the system can implement barb structures that can transition between protruding and non-protruding modes. For instance, during insertion/delivery of the system, the barbed structures can be non-protruding, but then after positioning of the system relative to the user's body is complete, the barbed structures can be transitioned into a protruding operation mode (e.g., upon mechanical activation with an actuator of the system, upon activation triggered by hydration from the user's body, using any other suitable activation trigger, etc.).

In one variation, the retention mechanism 140 includes an expandable feature, such as a medical balloon, wherein the retention mechanism 140 engages with tissue of the user through compressive forces (e.g., when the balloon is inflated with fluid) of the expandable feature against surrounding tissue of the user. In a specific example, the expandable feature is inserted into tissue of the user in a deflated state and then inflated once a nearby sensor has been appropriately located within the user. In a second specific example, the expandable feature is retained within a vessel (e.g., lumen) of the user.

In another variation, the retention mechanism 140 is partially or fully retained in tissue of the user through surface properties of the surrounding tissue (e.g., natural adhesion properties of subcutaneous tissue, adhesion properties of the cortical surface of brain tissue, etc.).

In a fourth variation, the retention mechanism 140 secures a sensor 120 and/or coupling subsystem 130 to a head region of the user through non-contact forces, such as magnetic forces. In a specific example, the retention mechanism includes a pair of magnets, a first of the pair of magnets arranged internal to the user and a second of the pair of magnets, the second magnetically attracted to the first, at an offset from the first of the pair of magnets (e.g., on an external surface of the user's scalp). This can function to protect tissue of the user, enable a mechanically adjustable system (e.g., by moving an external magnet), or can have any other suitable functionality.

### 3.5 System—Sensor Interface 150

The biosignal sensor subsystem 110 can include a set of one or more sensor interfaces 150, which individually or collectively function to electrically connect one or more of the set of sensors 120 to an electronics subsystem 160. Additionally or alternatively, the set of sensor interfaces 150 can function to mechanically connect one or more of the set of sensors 120 to the electronics subsystem 160, to the coupling subsystem 130, or to any other part of the system 100.

The set of sensor interfaces 150 can be electrically connected to one or more sensors 120, wherein electrical activity (e.g., EEG signals, brain waves, etc.) is received at the set of sensor interfaces 150 from the sensor channel(s). The set of sensor interfaces 150 can further be mechanically connected to one or more sensors 120, electrically and/or mechanically connected to the coupling subsystem 130, coupled to the user (e.g., arranged on skin, subcutaneous), or otherwise coupled to the system 100.

The set of sensor interfaces 150 functions to preprocess a set of signals from the set of sensors 120, prior to further processing and transmission at the electronics subsystem 160. Preferably, each of the sensor interfaces 150 comprises a pre-gain AC coupling and level shift coupled to an amplifier, and a post-gain AC coupling and level shift. The pre-gain AC coupling and level shift function to block direct current (DC) signals and shift signals from the set of sensors closer to a mid-rail voltage provided by a mid-rail generator of the electronics subsystem 160, in order to provide an approximately equal dynamic range with each polarity. As such, the pre-gain AC coupling and level shift preferably comprise a resistor-capacitor network which also functions as a high-pass filter with a minimum signal frequency (e.g., 0.159 Hz, below 0.2 Hz, below 1 Hz, between 0.01 and 0.5 Hz, above 1 Hz, etc.) that effectively blocks DC signals. The resistor-capacitor network is preferably coupled to an amplifier, whose feedback network (e.g., feedback resistor) gives it a suitable gain (e.g., 50). Furthermore, in order to limit a high frequency response and to avoid excessive phase shift due to the amplifier, the feedback resistor can be coupled to a capacitor in parallel to create a low-pass filter (e.g., a low pass filter that starts high-frequency roll-off at 80 Hz). The amplifier can be further coupled to a capacitor in series with a gain resistor, in order to prevent amplification of DC signals. A high gain resulting from amplification of a biosignal will result in an offset at the amplifier output; thus, the post-gain AC coupling and level shift functions to restore signal balance to the mid-rail voltage. In one variation, the post-gain AC coupling and level shift comprises a second high-pass resistor-capacitor network, but can comprise any other suitable element(s). The set of sensor interfaces 150 can additionally or alternatively comprise any suitable element or combination of elements for preprocessing of signals from the set of sensors 120.

The set of sensor interfaces 150 preferably includes one sensor interface 150 per sensor 120 such that each sensor has a dedicated sensor interface. Alternatively, in some variations, several sensors are multiplexed through a single sensor interface or a single sensor interacts with several sensor interfaces (e.g., to transmit a signal to multiple locations). One or more sensor interfaces 150 is preferably arranged proximal to one or more sensors, but can additionally or alternatively be centrally located with respect to a set of multiple sensors 120.

In one variation, the set of sensor interfaces are arranged subcutaneously within the user and directly electrically connected to the set of sensors 120 (e.g., via the coupling subsystem 130).

In a second variation, the set of sensor interfaces 150 are arranged supracutaneously. In a first specific example, the set of sensor interfaces 150 are electrically connected to the set of sensors 120 through transdermal terminals of the coupling subsystem 130. In a second specific example, the set of sensor interfaces 150 are electromagnetically connected to subdermal terminals of the coupling subsystem 1300.

### 3.6 System—Housing 152

The biosignal sensor subsystem 110 can include any number of housings (e.g., encapsulations, encapsulation housings, etc.) 152, which function to protect one or more sensors 120 or sensor interfaces 150 (e.g., from fluid ingress), mechanically connect a retention mechanism 140 (e.g., barbed structure) to a coupling subsystem 130, protect tissue of the user from electrical components of the system 100, or any other suitable function.

In variations of the system 100 having multiple housings 152, each of the housings preferably corresponds to each of the sensors 120 and functions to couple one or more retention mechanism to a region proximal to (e.g., adjacent to, on either side of, etc.) each of the sensors 120. Additionally or alternatively, each retention mechanism 140 can include its own housing (e.g., to secure the retention mechanism to a coupling subsystem), each coupling subsystem 130 can include one or more housings, or any number of housings can be arranged in any suitable arrangement. Each of the housings 152 is preferably constructed from a non-conductive material (e.g., polymer) but can alternatively be partially or fully constructed from any or all of: a conductive material, biocompatible material, biodegradable material, insulative material, or any other suitable material. The housing 152 can be in the form of a sleeve (e.g., shrink-wrapped sleeve, plastic tubing, etc.) or cylinder, partial sleeve or cylinder, shell, panel, box, or can have any other suitable form factor.

In one variation, one or more retention mechanisms 140 includes a housing 152 (e.g., encapsulation sleeve), where in the housing 152 functions to secure the retention mechanism 140 to the coupling subsystem 130 as well to at least partially encapsulate one or more sensors. In one specific example, the housing 152 at least partially defines an exposed contact region of a sensor 120.

In a second variation, the housing 152 can define one or more retention mechanisms. In a specific example, for instance, the housing can include a sleeve with barbed protrusions, wherein the barbed protrusions act as a retention mechanism 140.

In a third variation, the retention mechanism 140 can include a housing 152, such as a retention mechanism 140 in the form of a balloon, wherein the balloon partially or fully encapsulates one or more sensors 120 and/or the coupling subsystem 130.

### 3.7 System—Electronics Subsystem

The electronics subsystem 160 functions to provide regulated power to the system 100, to facilitate detection of biosignals from the user by incorporating signal processing elements, to couple to additional sensors for comprehensive collection of data relevant to the user and/or the biosignals being detected, and to enable transmission and/or reception of data by the system 100. As such, the electronics subsystem 160 can comprise a power module, a control module, a signal processing module, a supplementary sensor module, and a data link. The electronics subsystem 110 can additionally or alternatively comprise any suitable element(s) to further facilitate power distribution and biosignal handling.

The electronics subsystem 160 is preferably arranged at a central location with respect to the rest of the system 100 such that the one or more coupling subsystems 130 (e.g., multiple branches of a coupling subsystem 130) can establish an electrical connection with the electronics subsystem 160. This can be at a head region (e.g., proximal to the frontal cortex, behind one or more ears, etc.), a lower neck region, a facial region (e.g., nasion region), or another region of the user's body (e.g., shoulder, arm, etc.). The electronics subsystem 160 can be implanted in the user (e.g., subcutaneously, transcutaneously, at the same depth as another element of the system, at a different depth than another element of the system, etc.) but can additionally or alternatively be arranged on an external surface of the user (e.g., adhered to the skin), integrated or placed in a user garment, held by a user, or arranged in any other suitable way. In further alternative or additional variations, the electronics subsystem 160 can be partially or fully arranged remote from the rest of the system 100 and/or from the user (e.g., through a wireless connection).

The electronics subsystem 160 can comprise discrete components, or can alternatively comprise dedicated single-chip modules. Furthermore, the electronics subsystem 160 can comprise off-shelf components or custom integrated circuits configured to perform signal processing (e.g., signal conditioning, signal amplification). Preferably, the electronics subsystem 160 provides adequate connection characteristics to accommodate high input impedances associated with the sensors 120 of the biosignal sensor subsystem 110 (e.g., in the event of poor coupling at a sensor-user interface), and preferably, is configured to support input impedances in the range of 1 mega-ohm to 1 giga-ohm. However, the electronics subsystem 140 can alternatively accommodate any suitable range of input impedances (e.g., below 1 mega-ohm, above 1 giga-ohm, etc.). The electronics subsystem 160 can additionally or alternatively comprise any other suitable element or combination of elements for providing regulated or unregulated power to the system 100 and/or controlling elements of the system 100. Furthermore, the electronics subsystem 160 can additionally or alternatively comprise any other suitable combination of elements for handling biosignal detection, biosignal detection, biosignal processing, and/or biosignal transmission, in a manner that provides sufficient sensitivity.

The system 100 preferably includes one onboard electronics subsystem but can additionally or alternatively include multiple onboard electronics subsystems 160, no onboard electronics subsystems 160, any number of off-board (partially or fully external) electronics subsystems 160 (e.g., in one or more user devices, hospital equipment, etc.), or any number or arrangement of electronics subsystems.

The electronics subsystem 160 preferably implements the operation modes described previously for the biosignal sensor subsystem 110 but can additionally or alternatively implement a subset of these operation modes (e.g., only on/off modes), an additional set of operation modes (e.g., additional stimulation operation modes), or any number and type of operation modes.

In one variation, the components and/or processing steps of the electronics subsystem are distributed among one or more sensor interfaces 150. In one specific example, for instance, each sensor interface 150 includes an electronics subsystem 160 such that most or all of the processing is done at the set of sensor interfaces 150.

In a second variation, the processing steps (e.g., pre-processing steps) of one or more sensor interfaces 150 are at least partially designated to the electronics subsystem 160.

In a third variation, a second electronics subsystem in a user device (e.g., mobile phone, tablet, computer, smart phone, etc.) performs some or all of the processing. In one example, for instance, a user device receives bioelectrical signals from a wireless communication module of a first electronics subsystem onboard the user (e.g., implanted), wherein, for instance, further processing (e.g., more complex processing) can be done at the user device. In another example, one or more operation modes can be designated at the user device (e.g., on an application executed on a user device), wherein the operation mode is communicated (e.g., wirelessly) to the onboard electronics subsystem 130.

In some variations, the electronics subsystem 160 comprises a printed circuit board (PCB) configured to provide a substrate and connections for elements of the electronics subsystem 160. In a specific example, the PCB is coated in a biocompatible material to facilitate subcutaneous insertion and long-term (e.g., permanent, semi-permanent) emplacement beneath the skin of the user. In this specific example, the electronics module 160 is preferably encapsulated, and includes a wireless communication module and inductive charging capability (e.g., a slave coil that can be inductively coupled to an external master coil to facilitate charging of the electronics subsystem).

### 3.8 System—Power Module

The electronics subsystem 160 preferably includes a power module 164, which functions to store and distribute energy in order to power the system 100. As such, the power module 164 preferably includes an energy storage device (e.g., battery, rechargeable battery, capacitor, supercapacitor, etc.) and can be configured to couple to an external charging module in variations wherein the energy storage device is rechargeable. The energy storage device is preferably a battery coupled to voltage regulation and power distribution circuitry and can be rechargeable or non-rechargeable. The power module 164 can be active or passive (e.g., only conducts signals out where a receiver reads and processes the signals). Additionally or alternatively, the power module 164 can be involved in harvesting any or all of; heat, motion, brain signals, and any other input.

In variations wherein the battery is rechargeable, the battery can comprise a lithium-ion polymer battery (e.g., specified at 3.7V at 480 mAh, with a charged voltage of 4.2V and a discharged voltage of 3.0V) but can alternatively comprise any other suitable rechargeable battery (e.g., nickel-cadmium, metal halide, nickel metal hydride, or lithium-ion). In one specific example, the system 100 includes a rechargeable secondary battery (e.g., back-up battery).

In variations wherein the battery is a non-rechargeable battery, the battery can comprise an alkaline battery or other non-rechargeable battery that can be replaceable to enhance modularity in the system 100. The battery (or other suitable energy storage device) can be configured to charge by a wired connection (e.g., stereo connection, universal serial bus connection, custom connection) or by a wireless connection (e.g., by inductive charging). Furthermore, the supply voltage during charging is preferably regulated (e.g., using a resettable or non-resettable fuse) to prevent an overvoltage situation; however, the supply voltage can alternatively be unregulated (e.g., the system can omit a fuse). The voltage of the battery is preferably detected and regulated in real-time using a voltage detector and a voltage regulator; however, the voltage of the battery can alternatively be detected and/or regulated in any other suitable manner. In one variation, a voltage regulator coupled to the battery uses a detected battery voltage as a metric to determine if the system 100 should be shut off or turned on (e.g., a battery voltage below a lower threshold results in system shut off and a battery voltage above an upper threshold results in system turn on). In other variations, any other suitable characteristic of the battery, such as a temperature of the battery, can also be monitored and/or regulated.

In one variation, the power module is at least partially arranged external to the system and/or user. In one example, for instance, the power module 164 includes an inductive charger. In a specific example for this, the power module 164 includes a slave coil that can be inductively coupled to an external master coil to facilitate charging of the electronics subsystem 160. The external master coil can be portable, attached to the system and/or a user, built into a pillow, a head garment (e.g., hat, headset, earphones, etc.), a car head rest, a chair back, or arranged in any other way.

### 3.9 System—Control Module

The electronics subsystem 160 can include a control module 166, wherein the control module 166 is preferably a programmable module coupled to the power module 164, wherein the control module 166 functions to control the system 100. The control module 166 preferably comprises a microcontroller, and is preferably configured to control powering of the system 100, handling of signals received by the system 100, distribution of power within the system 100, and/or any other suitable function of the system 100. The control module can be configured to perform at least a portion of the method described in U.S. Pat. No. 7,865,235, and/or U.S. Publication Nos. 2007/0066914 and 2007/0173733, which are each incorporated in their entirety by this reference. In other variations, the control module 166 can be additionally or alternatively be configured to enable or perform a portion of the methods described in U.S. application Ser. Nos. 13/903,806, 13/903,832, and 13/903,861, each filed on 28 May 2013, which are each incorporated in their entirety by this reference. In some variations, the control module 166 can be preconfigured to perform a given method, with the system 100 configured such that the microcontroller cannot be reconfigured to perform a method different from or modified from the given method. However, in other variations of the system 100, the microcontroller can be reconfigurable to perform different methods.

### 3.10 System—Signal Processing Module

The electronics subsystem 160 can include a signal processing module 168, which preferably comprises an amplifier, a filter, and an analog-to-digital converter (ADC) and can additionally comprise a multiplexer configured to multiplex signals from multiple sensor channels. As such, the signal processing module 168 functions to process detected and received biosignals from the set of sensors, in order to facilitate further processing and/or signal analysis. The signal processing module is preferably coupled to the sensor interface 150 and to the control module of the electronics subsystem, in order to facilitate handling of detected biosignals and signal processing.

The amplifier functions to amplify a detected biosignal, in order to facilitate signal processing by the system 100. The system can comprise any suitable number of amplifiers, depending upon the configuration of the amplifier(s) relative to other elements (e.g., multiplexers) of the electronics subsystem 140. In one variation, the amplifier is placed after a multiplexer in order to amplify a single output line. In another variation, a set of amplifiers is placed before a multiplexer, in order to amplify multiple input channels into the multiplexer. In yet another variation, the electronics subsystem 160 comprises amplifiers before and after a multiplexer, in order to amplify input and output lines of the multiplexer. The amplifier can also be coupled to a filter configured to suppress inter-channel switching transients (e.g., produced during multiplexing), and/or any other undesireable signals. In one example, the amplifier is coupled to a transient filter configured to dampen transients resulting from voltage potentials (e.g., voltage potentials of 3V) between consecutively selected multiplexer channels, thus reducing the settling time and improving a signal sampling rate. In other examples, the filter can be a low pass filter, a high pass filter, or a band pass filter configured to only allow passage of a certain range of signals, while blocking other signals (e.g., interference, noise) outside of the range of signals. The ADC functions to convert analog signals (e.g., biosignals detected by the set of sensors, amplified signals, filtered signals) into a digital quantization. The ADC can be characterized by any suitable number of bits, and in a specific example, is characterized by 16-bits, with only 14-bits being used. The ADC can also comprise an internal voltage reference. The electronics system 160 can comprise any suitable number of ADCs for conversion of analog signals (e.g., from multiple channels) into digital quantizations.

In variations of the electronics system 160 comprising a multiplexer, the multiplexer is preferably configured to receive multiple signals from the set of sensors 120 through a sensor interface 150 of the biosignal sensor subsystem 110, and to forward the multiple signals received at multiple input lines in a single line at the electronics subsystem 160. The multiplexer thus increases an amount of data that can be transmitted within a given time and/or bandwidth constraint. The number of input channels to the multiplexer is preferably greater than or equal to the number of output channels of the biosignal sensor subsystem, wherein a 2̂n relationship exists between the number of input lines and the number of select lines of the multiplexer (e.g., a multiplexer of 2̂n input lines has n select lines, which are used to select an input line to output). In a specific example, the biosignal sensor subsystem 110 comprises five channels with a spare channel, and the multiplexer comprises eight input lines (e.g. the multiplexer is an 8:1 multiplexer) with three parallel select lines. In the specific example, the multiplexer has a low-voltage switch on resistance of 2 ohms. The multiplexer can include a post-multiplexer gain (e.g., 10) in order to reduce capacitance values of front-end amplifiers coupled to the multiplexer; however, the multiplexer can alternatively not include any gain. The multiplexer can also include high frequency and/or low frequency limiting.

In some variations, the signal processing module is configured to determine a quality metric (e.g., contact quality metric, signal quality metric, etc.) of one or more of the set of sensors 120, which functions to determine whether or not there is a sufficient quality of contact (e.g., contact quality above a predetermined threshold) between the sensor(s) and the user. This can include any of the systems and methods (e.g., estimating a quality of contact based on an amplitude of a detected sensor signal corresponding to a predetermined square wave signal frequency provided by a hum-remover/square-wave generator) in U.S. application Ser. No. 14/447,326 filed 30 Jul. 2014, which is incorporated in its entirety by this reference, U.S. application Ser. No. 15/209,582 filed 13 Jul. 2016, which is incorporated in its entirety by this reference, or any other suitable systems and methods for determining a quality metric.

### 3.11 System—Data Link

The electronics subsystem 160 can include a data link 170, wherein the data link 170 is preferably coupled to the control module 166, and functions to transmit an output of at least one element of the electronics subsystem 140 to a mobile device (e.g., user device) or other computing device (e.g., desktop computer, laptop computer, tablet, smartphone, health tracking device); additionally or alternatively, the data link 170 can be coupled to the power module 164 or any other element of the system 100. Preferably, the data link 170 is a wireless interface; however, the data link can alternatively be a wired connection. In a first variation, the data link 170 can include a Bluetooth module that interfaces with a second Bluetooth module included in the mobile device or external element, wherein data or signals are transmitted by the data link to/from the mobile device or external element over Bluetooth communications. In an example of the first variation, the Bluetooth module comprises a 32 MHz crystal oscillator for radiofrequency transmissions, a 32.768 kHz crystal oscillator for standby operations, and common mode choke configured to reduce noise being conducted back into the system 100. The data link of the first variation can alternatively implement other types of wireless communications, such as 3G, 4G, radio, or Wi-Fi communication. The data link can include any elements configured for wireless communication, such as an antenna, WiFi chip, Bluetooth chip, or any other component, which can be integrated into a PCB of the electronics subsystem 160, arranged external to the system 100 or user, or otherwise arranged.

In one variation, data and/or signals are preferably encrypted before being transmitted by the data link, in particular, for applications wherein the data and/or signals comprise medical data. For example, cryptographic protocols such as Diffie-Hellman key exchange, Wireless Transport Layer Security (WTLS), or any other suitable type of protocol may be used. The data encryption may also comply with standards such as the Data Encryption Standard (DES), Triple Data Encryption Standard (3-DES), or Advanced Encryption Standard (AES).

Examples of the user device include a tablet, smartphone, mobile phone, laptop, watch, wearable device (e.g., glasses), or any other suitable user device. The user device can include power storage (e.g., a battery), processing systems (e.g., CPU, GPU, memory, etc.), user outputs (e.g., display, speaker, vibration mechanism, etc.), user inputs (e.g., a keyboard, touchscreen, microphone, etc.), a location system (e.g., a GPS system), sensors (e.g., optical sensors, such as light sensors and cameras, orientation sensors, such as accelerometers, gyroscopes, and altimeters, audio sensors, such as microphones, etc.), data communication system (e.g., a WiFi module, BLE, cellular module, etc.), or any other suitable component.

### 3.12 System—Additional Components

The system 100 can further include any number of insertion components (e.g., surgical tools) configured to implant any or all of the system 100 within or on the user, such as in accordance with the method 200 described below or any other suitable method. These can include any or all of: needles, guide catheters, scalpels, sutures, bandages, stitching, stereoscopic equipment, or any other tool or machinery.

The system 100 can further include or be coupled to additional sensor subsystems configured to capture data related to other biological processes of the user and/or the environment of the user. As such, the biosignal sensor subsystem can comprise optical sensors to receive visual information about the user's environment, GPS elements to receive location information relevant to the user, audio sensors to receive audio information about the user's environment, temperature sensors, sensors to detect MEG impedance, electromagnetic fluctuations of any kind, or galvanic skin response (GSR), sensors to measure respiratory rate, and/or any other suitable sensor.

The system 100 can further include a payload. The payload can include one or more elements of the system 100 as described, the entire system, or any or all of: an output element (e.g., light, microphone, etc.), a supplementary processing system, one or more electrical connectors (e.g., accessories plug-in), and any other component. The payload can have a weight and/or mass above a predetermined threshold (e.g, above 0.1 grams, above 0.5 grams, above 2 grams, etc.) and/or below a predetermined threshold (e.g., less than 5 grams, less than 10 grams, etc.).

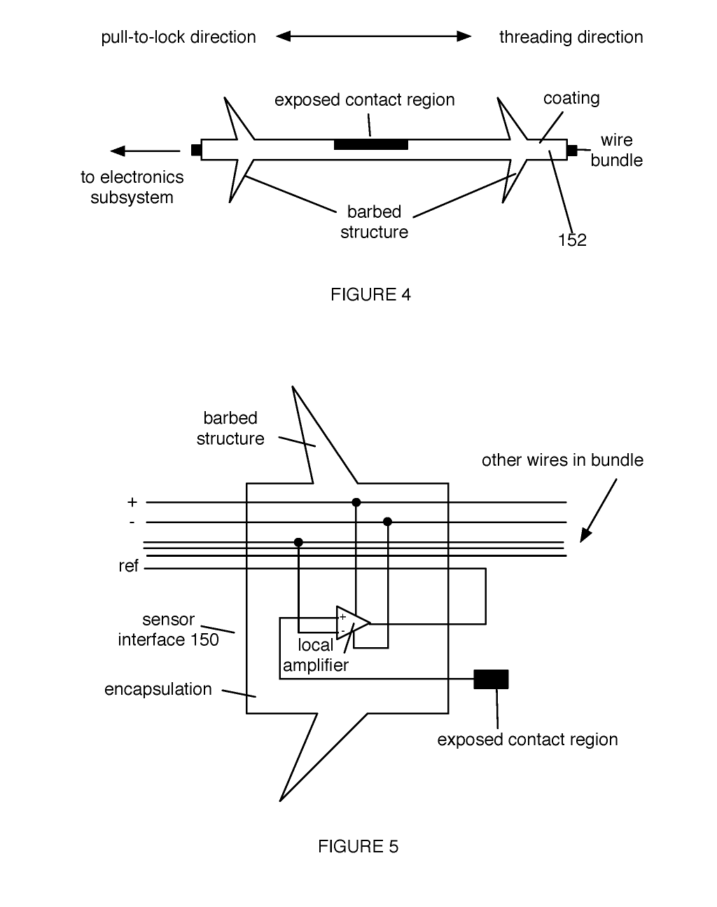

In a variation of the system, as shown in FIG. 2, the set of sensors forms a mesh of sensors radiating from a central wireless electronics module. The wireless electronics module is placed unobtrusively at a central location (e.g., at the base of the rear of the user's head). Each sensor is affixed to a conductive suture-like member, and each conductive suture-like member can be attached to (e.g., configured to conduct signals from) a single sensor or multiple sensors. The sensors are coupled to the user by inserting the conductive suture-like members beneath the skin of the user's scalp, and threaded across the user's head for any suitable distance required to position the sensor(s) at the desired locations relative to the brain of the user. At one or more positions along each conductive suture-like member (e.g., at each sensor position, at one position, at a plurality of positions uncorrelated with the sensor positions, etc.), the member defines a barbed structure configured to lock into soft tissues. In the first specific example, locking of members into respective tissues regions is preferably initiated upon pulling of a member in the opposing direction to the threading direction (as shown in FIG. 4), but locking can additionally or alternatively be performed absent any application of tension to the member. The barbed structure(s) preferably emplace the sensor(s) at the desired position(s) and reduce migration and/or other movement relative to the initial position. In some variations, slack (e.g., additional length) can be allowed to remain in one or more of the suture-like members after insertion to allow for flexibility of the sensor mesh upon normal movement (e.g., movement of the user during the course of daily activities such as walking, sleeping, exercising, etc.). The central wireless electronics module is preferably encapsulated subcutaneously, and is preferably configured to wirelessly communicate with an external receiver (e.g., to transmit biosignals detected at the sensors). However, the electronics module can, in related examples, have transdermal connection points that provide an interface for wired electronic signal (e.g., data, power) transmission.

In a second variation of the system, the system 100 includes a set of sensors 120, wherein the sensors form a wireless sensor network (e.g., no coupling subsystem), each sensor configured to wirelessly communicate with an electronics subsystem and to be charged through inductive charging. In a first specific example, the electronics subsystem is implanted in the user (e.g., in the user's scalp). In an alternative example, the electronics subsystem is arranged external to the user.

In a third variation of the system, the set of sensors 120 comprises a set of passive sensors, wherein signals from the passive sensors are detected, measured, and/or processed external to the user (e.g., through placing a conductive medium proximal to the sensor but external to the user).

### 4. Method

The method 200 functions to insert any or all of the system 100 within or on a head region of the user, preferably a relatively superior head region (arranged superior to any or all of the frontal lobe, temporal lobe, occipital lobe, parietal lobe(s), cerebellum, brain, cortex, etc.), but additionally or alternatively any other head or body region of the user. The method 200 is preferably configured to install the system 100 without tension, wherein the system 100 can, for instance, include slack once installed, but can be configured to install the system in any suitable way. Additionally or alternatively, the method 200 can function to process one or more signals received from the user, perform any common surgical insertion procedure (e.g., suturing procedure, minimally invasive procedure, etc.) or perform any of the methods incorporated by reference. The variations and examples of the method 200 can be performed alone, in combination, or in any other arrangement.

The steps of the method are preferably performed by a medical professional (e.g., surgeon, physician, nurse, etc.) but can alternatively be performed by a user (e.g., when system 100 is applied external to the user, such as on the user's skin), technician (ex. in an outpatient procedure), with a robot (e.g., automatically), by a physician or instructor in a teaching or instructional setting, or any other entity or system. In a specific example, the sensor installation method can be similar to that used for barbed suture lifts, wherein the electrical terminals can be attached to the coupling subsystem 130 end after sensor installation or otherwise installed.

The method can include step S210: inserting a payload (e.g., system 100, sensors 120, etc.) into a user, which functions to gain access to head region (e.g., access subcutaneous tissue, transcutaneous tissue, brain matter, skull, skin, etc.) of the user. Preferably the biosignal sensor subsystem 110 in inserted into the user but the electronics subsystem 160 and/or any other element can additionally or alternatively be inserted in S210. Step S210 is preferably performed first but can additionally or alternatively be performed after another step (e.g., after determining a location for one or more sensors, preparing the insertion site of the user (e.g., disinfecting), etc.) and/or performed multiple times throughout the method. In variations of the system having multiple biosignal sensor subsystems, the biosignal sensor subsystems can be inserted through the same insertion site (e.g., simultaneously, sequentially, in response to the completion of placing another biosignal sensor subsystem, etc.), through different insertion sites (e.g., simultaneously, sequentially, in response to the completion of placing another biosignal sensor subsystem, etc.)—which can function to locate different biosignal sensor subsystems to different head regions—and/or inserted in any other way.

Step S210 is preferably performed based on the determination of one or more desired sensor head region locations (e.g., brain lobe, set of coordinates, depth of insertion, etc.), which can be determined through any or all of: assessing the user's medical history (e.g., locating a region associated with a user neuropathology), assessing the user's anatomy (e.g., determining anatomical locations in a set of user medical images), determining a desired location based on aggregated data (e.g., averaged coordinates from a dataset), or any other suitable way of determining suitable sensor locations. Alternatively, one or more sensors can be placed randomly or without prior planning.

Step S210 can be performed by making an incision (e.g., to a transcutaneous depth, to a subcutaneous depth, etc.), making a puncture hole (e.g., with a needle, cannula, etc.), applying compression (e.g., affixing a sensor to the skin of a user), and/or any other method of engaging with the user. In some variations S210 further includes any or all of: drilling a hole (e.g., through skull), excising/removing brain coverings (e.g., dura mater, pia mater, etc.), inserting any or all of the electronics subsystem into the user, inserting any surgical components (e.g., sheath, introducer needle, scalpel, etc.) into the user, and/or performing any other action in order to access the head region of interest.

Step S220 includes navigating any or all of the payload through tissue of user, which functions to advance the payload (e.g., set of sensors 120) to a desired head region of the user. The payload can include any element of the system 100 (e.g., biosignal sensor subsystem, sensor, housing, sensor interface, etc.), multiple elements of the system 100 (e.g., two biosignal sensor susbsystems), the entire system 100, or an extraneous payload (e.g., therapeutic element, drug delivery, etc.).

S220 is preferably performed after S210 but can alternatively be performed in parallel with step S210, in place of step S210, or at any other point in the method. S2320 is preferably performed in conjunction with a covering (e.g., guide catheter, sheath, etc.) over one or more retention mechanisms, wherein the covering is configured to prevent coupling of the retention mechanism with the user prior to locating the sensor at its desired location S230. Alternatively, however, in some variations the retention mechanism (e.g., balloon, set of magnets, etc.) is deployed while exposed to the user. The covering can further function to reduce friction during navigation, protect one or more components from biodegradation, or perform any other suitable function.

Step S220 can be performed along any suitable trajectory. In one variation, the payload is navigated along a shortest trajectory (e.g., straight trajectory) to minimize disruption to tissue of user, minimize material costs, or for any other purpose. In another variation, the payload is navigated sinuously through the head region, which can function to accommodate slack in the coupling subsystem (e.g., one or more wires of a wire bundle), construct a wide path for future navigation steps, permit shifting of the system, or for perform any other purpose. S220 is preferably performed through pushing but can additionally or alternatively be performed through pulling, through the influence of gravity (e.g., placed in a long incision), or in any other way. The navigation can be performed with any number of navigation tools and methods. In some variations, external systems are used to assist with navigation. In one example, for instance, a set of magnets external to the user (e.g., on the scalp of the user) can be used to move a system 100 within the user (e.g., move a magnetic tip of the coupling subsystem through the tissue of the user). In another variation, any number of imaging methods for navigation (e.g., stereotactic guidance, imaging with contrast agents, etc.) can be used during this step or at any point during the method S200.

Step S230 includes locating any or all of the payload within tissue of a user, which functions to ensure proper placement of the payload (e.g., sensor 120) within or on the user. S230 is preferably performed after or in parallel with S220 but can alternatively be performed multiple times throughout the method 200 (e.g., once per sensor) or at any other point throughout the method. Step S230 is preferably performed with imaging guidance, such as described in Step S220, but can alternatively be performed without guidance (e.g., based on physician experience, based on tactile qualities such as relative resistance or stiffness while advancing the system 100, randomly).

Step S240 includes securing the payload, wherein the payload includes a set of sensors, to the user. Step S240 functions to secure the system 100 to the user and maintain desired sensor placement (e.g., exact sensor placement, placement within a gradual shift threshold, flexible placement, etc.). S240 is preferably performed as a permanent step (e.g., to ensure final placement of sensors 120) but can additionally or alternatively be performed as a temporary step (e.g., to progress along a trajectory such as through inflation and deflation of a balloon, deployment and retraction of a protrusion, etc.) during the method S200. Step S240 preferably includes exposing a retention mechanism to the surrounding tissue of the user and engaging the retention mechanism with the surrounding tissue. Exposing the retention mechanism can be performed by pulling a covering (e.g., sheath, cannula, etc.) distally from the sensor location (e.g., toward the insertion point), pushing the retention mechanism out of a covering (e.g., holding the covering at a fixed position), applying a force and/or torsion to free the retention mechanism from a covering, or through any other suitable action. Engaging the retention mechanism is preferably performed through a pulling or a pushing action, such that an angled protrusion (e.g., barb) engages with (e.g., pierces, hooks on, locks into) tissue of the user. Additionally or alternatively, engaging the retention mechanism can be performed through a torsion action (e.g., engaging a corkscrew with tissue), deploying a fluid (e.g., into a balloon), applying compression (e.g., to adhere a retention mechanism to tissue), applying tension, or through any other action. In variations of the system 100 without a retention mechanism, the method S200 can be performed in absence of any or all of step S240 and/or can replace S240 with any suitable step, such as deploying a sensor (e.g., from a covering) to a head region of the user.

Step S240 can further include a sub-step S242: engaging a second retention mechanism with a user, which can function to incorporate slack into the system. In one variation for instance, a second retention mechanism is deployed separately from the first retention mechanism with a predetermined amount of slack in the coupling subsystem (e.g., 5 mm of wire, 10 mm of wire, 15 mm of wire, greater than 1 mm of wire, etc.), wherein slack refers to an arrangement wherein the length of the coupling subsystem (e.g., wire bundle) between a first sensor point (e.g., actual sensor location, desired sensor location, sensor coordinates, etc.) and a second sensor point is greater than a distance (e.g., shortest distance, specific trajectory, arc length, etc.) between the first and second points. Alternatively, the system can be positioned with no slack, in tension, or in any other arrangement. Step S242 is preferably performed after S240 but can alternatively be performed in parallel to S240.

In one variation of S240 (e.g., as shown in FIG. 7), a covering is advanced (e.g., pulled toward an insertion point) prior to coupling. In one example, a sheath encircling the retention mechanism and coupling subsystem is pulled toward the insertion point once the sensor is proximal to its desired location such that the retention mechanism is exposed to the head region of the user and configured to be engaged with the tissue of the head region.

The method can include Step S250: removing an extraneous component (e.g., insertion component) from the user, which functions to remove temporary elements of the method, such as surgical tools, temporary coverings, introducer needles, etc.

Step S254 includes placing an electronics subsystem, which functions to couple the electronics subsystem to the user and/or electrically connect the electronics subsystem with other elements of the system 100 (e.g., coupling subsystem, sensor interface, sensor, etc.). Step S254 is preferably performed after all the sensors and retention mechanisms have been placed but can additionally or alternatively be performed in parallel with any other step (e.g., S240), at the beginning of the method S200, in parallel with S210, not at all (e.g., when processing is done through the sensor interfaces), or at any other point in the method. The electronics subsystem 160 can be placed proximal to (e.g., within, adjacent to, etc.) the insertion point of S210, at another region of the user (e.g., nasion region, lower neck region, etc.), external to the user (e.g., adhered to the skin, adhered to a garment, in a pocket unit, etc.), or at any other location. The electronics subsystem 160 can be placed at the same depth as another element of the system 100, any suitable depth (e.g., transcutaneous, subcutaneous, etc.), placed on an external surface (e.g., with an adhesive), or at any other location.

The method S200 can include Step S256: securing and or repairing the insertion site(s) after the payload has been inserted, which functions to fully secure the system to the user and prevent infection. Step S256 can involve stitching, suturing, applying a wound closure adhesive (e.g., skin glue), or any other step. Alternatively, the insertion site can heal naturally (e.g., small insertion point).

The method S200 can additionally or alternatively include any other method step, performed at any time during the method. The method can additionally or alternatively be performed in absence of any of the steps described. The the steps can be performed in any order, as well as individually, in parallel, multiple times, or in any other way at any time.

Although omitted for conciseness, the preferred embodiments include every combination and permutation of the various system components and the various method processes, wherein the method processes can be performed in any suitable order, sequentially or concurrently. Variations of the system and method can be performed individually, together, or in any combination.

As a person skilled in the art will recognize from the previous detailed description and from the figures and claims, modifications and changes can be made to the preferred embodiments of the invention without departing from the scope of this invention defined in the following claims.

1. A system for detecting a biosignal at a subcutaneous head region of a user, the system comprising:
a biosignal sensor subsystem, the biosignal sensor subsystem comprising:
a coupling subsystem, the coupling subsystem defining a series of exposed contact regions along a length of the coupling subsystem;
a set of barbed structures connected to the coupling subsystem;
a sensor interface electrically connected to the series of exposed contact regions;
an electronics subsystem electrically connected to the biosignal sensor subsystem, the electronics subsystem comprising:
a wireless communication module; and
an inductive charging system electrically connected to the wireless communication module.

2. The system of claim 1, wherein one or more barbed structures is arranged proximal to each of the set of exposed contact regions, further wherein each of the set of barbed structures has the same orientation.

3. The system of claim 1, wherein the coupling subsystem comprises at least one of: a wire bundle, the wire bundle comprising a plurality of wires; and a flexible printed circuit.

4. The system of claim 1, wherein the sensor interface comprises a high-pass filter having a predetermined minimum signal frequency, the sensor interface further comprising an amplifier coupled to the high-pass filter.

5. The system of claim 1, wherein the coupling subsystem comprises poly(3,4-ethylenedioxythiophene).

6. The system of claim 4, further comprising a plurality of the wire bundles, wherein each of the plurality of wire bundles extends radially outward from the electronics subsystem.

7. The system of claim 1, wherein each of the exposed contact regions comprises a length between two and three millimeters.

8. The system of claim 1, wherein each of the barbed structure is offset from each of the exposed contact regions.

9. A system for detecting a biosignal at a subcutaneous head region of a user, the system comprising:
a coupling subsystem comprising a series of sensors arranged along a length of the coupling subsystem;
a set of barbed structures connected to the coupling subsystem and arranged proximal to each of the set of sensors;
a sensor interface electrically connected to the series of sensors;
an electronics subsystem comprising a wireless communication module and a power module, the electronics subsystem electrically connected to the coupling subsystem.

10. The system of claim 9, wherein the sensor interface comprises a high-pass filter having a predetermined minimum signal frequency and an amplifier coupled to the high-pass filter.

11. The system of claim 10, wherein the predetermined minimum signal frequency is between 0.1 and 0.5 Hz.

12. The system of claim 9, further comprising a plurality of the coupling subsystems, each of the coupling subsystems comprising a wire bundle, wherein the electronics subsystem in an installed configuration is arranged subcutaneously at a second head region of the user, the second head region arranged inferior to the first head region, and wherein each of the plurality of coupling subsystems extends radially outward from the electronics subsystem.

13. The system of claim 9, further comprising an optical sensor electrically connected to the electronics subsystem.

14. A method for delivering a biosignal detection system to a head region of a user, the method comprising:
inserting a first end of the biosignal detection system into a first head region of the user, the biosignal detection system comprising a coupling subsystem, a set of payloads connected to the coupling subsystem, and a set of barbs arranged proximal to the plurality of payloads;
advancing a first payload in a first direction to a second head region of the user, the second head region arranged superior to the first head region; and
advancing the first payload in a second direction opposing the first direction, wherein advancement in the second direction engages at least one of the set of barbs with the second head region of the user.

15. The method of claim 14, further comprising:
advancing a second payload in the first direction toward a third head region of the user, the third head region arranged anterior to the second head region; and
advancing the second payload in the second direction, wherein advancement in the second direction engages at least one of the set of barbs with the third head region of the user.

16. The method of claim 15, wherein each of the first and second payloads are inserted to a depth of at least 1450 microns beneath a scalp of the user.

17. The method of claim 14, wherein the first payload is inserted with slack in the coupling subsystem.

18. The method of claim 14, wherein the second head region is arranged superior to at least part of one of a frontal and parietal lobe region of the user.

19. The method of claim 14, further comprising implanting an electronics subsystem proximal to the first head region of the user.

20. The method of claim 14, wherein the payload comprises an electroencephalography electrode.
